(19) United States
(12) Patent Application Publication
(10) Pub. No.: US 2015/0374239 A1
(43) Pub. Date: Dec. 31, 2015

(54) OBJECT INFORMATION ACQUIRING APPARATUS AND CONTROL METHOD OF OBJECT INFORMATION ACQUIRING APPARATUS

(71) Applicant: CANON KABUSHIKI KAISHA, Ohta-ku, Tokyo (JP)

(72) Inventors: Hiroshi YAMAMOTO, Kawasaki-shi (JP); Yukio FURUKAWA, Kyoto-shi (JP)

(21) Appl. No.: 14/767,080

(22) PCT Filed: Feb. 27, 2014
(86) PCT No.: PCT/JP2014/001078

§ 371 Date: Aug. 11, 2015

(30) Foreign Application Priority Data
Mar. 5, 2013 (JP) 2013-043337

(51) Int. Cl. A61B 5/00

(52) U.S. Cl. CPC A61B 5/0095; A61B 5/708; A61B 2576/00

(57) ABSTRACT

An object information acquiring apparatus, comprises a light irradiation unit that irradiates light onto an object and can change an emission direction of the light; a scanning mechanism that moves the light irradiation unit along a first axis; a probe that receives an acoustic wave generated by light irradiated onto the object; a processing unit that generates characteristic information inside the object based on the acoustic wave received by the probe; and a control unit that controls a position of the light irradiation unit on the first axis, and the emission direction of the light emitted from the light irradiation unit, wherein the control unit determines the emission direction of the light emitted from the light irradiation unit based on the position of the light irradiation unit on the first axis.

### TECHNICAL FIELD

The present invention relates to an object information acquiring apparatus and a control method thereof, and more particularly to a technique to improve the intensity of a signal generated in an object.

### BACKGROUND ART

Many proposals have been made thus far on techniques to non-invasively image a tomographic image of an area inside an object. One such technique is photoacoustic tomography (PAT), which acquires biological functional information using light and an ultrasound wave.

Photoacoustic tomography is a technique where pulsed light generated in a light source (hereafter called “measurement light”) is irradiated onto an object, an acoustic wave (typically an ultrasound wave) generated by the light being absorbed inside the object is received and analyzed, and the internal tissue of the object is visualized. By analyzing the received acoustic wave, an initial sound pressure distribution due to a light absorber inside the object can be acquired. Further, information related to the optical characteristics, such as an absorption coefficient inside the object, can be acquired by performing an arithmetic operation on the initial sound pressure distribution while considering the distribution of the light.

The sound pressure of the acoustic wave generated in the object in PAT is in proportion to the quantity of local light that reaches the light absorber. Therefore in order to acquire accurate information inside an organism, the quantity of the measurement light that is irradiated onto the object must be increased.

An apparatus according to Non Patent Literature 1 is an example of an apparatus that diagnoses breast cancer using PAT techniques. In this apparatus, a breast of a test subject is compressed and held by two holding members, so as to secure the quantity of the measurement light that reaches an area inside the breast.

On the other hand, for an accurate diagnosis of breast cancer, not only the breast but also an area including the chest wall portion on the root of the breast must be observed. In other words, when breast cancer is diagnosed using a PAT apparatus, the quantity of the measurement light to be irradiated not only onto the breast but also onto an area around the chest wall must be increased, in order to accurately obtain information on an area around the chest wall.

A photoacoustic imaging apparatus according to Patent Literature 1 is an invention to solve this problem. According to this apparatus, a light irradiation unit is disposed so that light irradiated onto an object is directed to the chest wall direction, whereby the quantity of measurement light to be irradiated onto the chest wall portion is increased.

### CITATION LIST

### Patent Literature

[PTL 1] Japanese Patent Application Laid-Open No. 2011-183057

### Non-Patent Literature

[NPL 1] Susanne E. et al., “First clinical trials of the Twente photoacoustic mammoscope (PAM)”, Proceedings of the SPIE, Vol. 6629, pp. 662917, 2007

### SUMMARY OF INVENTION

### Technical Problem

However in the case of the apparatus according to Patent Literature 1, the measurement light must be emitted from the entire surface of the holding member contacting the breast, hence an enormous number of light irradiation units are required, and such an apparatus configuration becomes impractical. To create a practical apparatus configuration, it is necessary to dispose a compact light irradiation unit for the object, and move the light irradiation unit while irradiating the measurement light onto the breast that is being held. However in the case of such a measurement apparatus which moves the light irradiation unit for scanning, a sufficient quantity of light cannot be irradiated onto the area around the chest wall, since the measurement light vertically enters the object. In other words, the photoacoustic signal generated near the chest wall cannot be accurately acquired.

With the foregoing in view, it is an object of the present invention to provide an object information acquiring apparatus that can accurately acquire a photoacoustic signal generated inside an object.

### Solution to Problem

The present invention in its one aspect provides an object information acquiring apparatus, comprises a light irradiation unit that irradiates light onto an object and can change an emission direction of the light; a scanning mechanism that moves the light irradiation unit along a first axis; a probe that receives an acoustic wave generated by light irradiated onto the object; a processing unit that generates characteristic information inside the object based on the acoustic wave received by the probe; and a control unit that controls a position of the light irradiation unit on the first axis, and the emission direction of the light emitted from the light irradiation unit, wherein the control unit determines the emission direction of the light emitted from the light irradiation unit based on the position of the light irradiation unit on the first axis.

The present invention in its another aspect provides a control method of an object information acquiring apparatus including a light irradiation unit that irradiates light onto an object and can change an emission direction of the light, and a probe that receives an acoustic wave generated by light irradiated onto the object, the control method comprises a moving step of moving the light irradiation unit along a first axis; a control step of determining the emission direction of the light based on a position of the light irradiation unit on the first axis; a receiving step of receiving an acoustic wave by the probe; and a processing step of generating characteristic information inside the object based on the received acoustic wave.

### Advantageous Effects of Invention

The present invention can provide an object information acquiring apparatus that can accurately acquire a photoacoustic signal generated inside an object.

Further features of the present invention will become apparent from the following description of exemplary embodiments with reference to the attached drawings.

### BRIEF DESCRIPTION OF DRAWINGS

FIG. 1A and FIG. 1B are set of diagrams depicting a configuration of a photoacoustic measurement apparatus according to Embodiment 1;

FIG. 2 is a diagram depicting a light irradiation unit according to Embodiment 1;

FIG. 3 is a diagram depicting a scanning route of the light irradiation unit according to Embodiment 1;

FIG. 4 is a flow chart depicting a measurement according to Embodiment 1;

FIG. 5A and FIG. 5B are set of diagrams depicting a configuration of a photoacoustic measurement apparatus according to Embodiment 2;

FIG. 6A and FIG. 6B are set of diagrams depicting a structure of a light irradiation unit according to Embodiment 2;

FIG. 7 is a diagram depicting a scanning route of a light irradiation unit according to Embodiment 3;

FIG. 8 is a flow chart depicting a measurement according to Embodiment 3; and

FIG. 9A is a diagram for describing an effect of an example.

FIG. 9B is a diagram for describing an effect of an example.

FIG. 9C is a diagram for describing an effect of an example.

### DESCRIPTION OF EMBODIMENTS

### Embodiment 1

Embodiment 1 of the present invention will now be described in detail with reference to the drawings.

A photoacoustic measurement apparatus according to Embodiment 1 is an apparatus that images information inside an organism (an object) by irradiating measurement light onto the object, and receiving and analyzing an acoustic wave generated inside the object due to the measurement light.

<System Configuration>

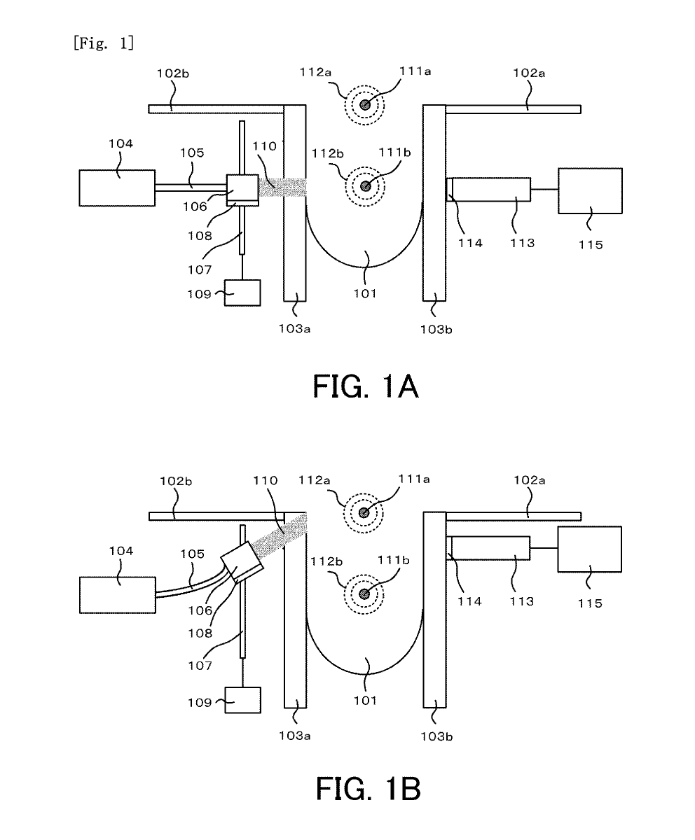

A configuration of the photoacoustic measurement apparatus according to the present embodiment will be described first with reference to FIG. 1A.

The photoacoustic measurement apparatus according to Embodiment 1 has chest wall support units 102a and 102b, a holding member 103, a light source 104, a light transmission unit 105, a light irradiation unit 106, a scanning mechanism 107, a rotation mechanism 108, a control unit 109, a probe 113 and a processing unit 115. The holding member 103 is constituted by a movable holding member 103a and a fixed holding member 103b.

Although not included in the apparatus, the reference sign 101 in FIG. 1A is an object, the reference sign 110 is a measurement light, the reference sign 111a is a first light absorber, the reference sign 111b is a second light absorber, the reference sign 112a is a first acoustic wave, the reference sign 112b is a second acoustic wave, and the reference sign 114 is an acoustic matching agent.

Measurement is performed in a state where a breast of a test subject is inserted into an opening created in the chest wall support unit 102 (in the present invention a test subject support member), and the inserted breast is held between the movable holding member 103a and the fixed holding member 103b.

First pulsed light emitted from the light irradiation unit 106 is irradiated onto the object 101 through the movable holding member 103a. When a part of the energy of light that propagates inside the object is absorbed by a light absorber, such as blood, an acoustic wave is generated by the thermal expansion of the light absorber. The acoustic wave generated inside the object is received by the probe 113 through the fixed holding member 103b, and is analyzed by the processing unit 115. Since the analysis result is outputted as an image expressing the characteristic information inside the object, the photoacoustic measurement apparatus according to this embodiment can also be called an “object information acquiring apparatus.”

Now each unit constituting the photoacoustic measurement apparatus according to this embodiment will be described.

<<Light Source 104>>

The light source 104 generates pulsed light. The light source is preferably a laser light source in order to obtain high power output, but a light emitting diode, a flash lamp or the like may be used instead of a laser. If a laser is used for the light source, various lasers including a solid-state laser, a gas laser, a dye laser and a semiconductor laser can be used. Irradiation timing, waveform, intensity or the like are controlled by a light source control unit (not illustrated). The light source control unit may be integrated with the light source.

To effectively generate a photoacoustic wave, light must be irradiated for a sufficiently short period of time in accordance with the thermal characteristics of the object. If the object is an organism, the pulse width of the pulsed light generated from the light source is preferably about 10 to 50 nanoseconds. The wavelength of the pulsed light is preferably a wavelength which allows the light to propagate inside the object. In concrete terms, a wavelength of 600 nm or more and 1100 nm or less is preferable if the object is an organism.

<<Light Transmission Unit 105>>

The light transmission unit 105 guides the pulsed light generated in the light source 104 to the object 101. In concrete terms, an optical member constituted by an optical fiber, a lens, a mirror, a diffusion plate or the like is used so as to acquire a desired beam shape and light intensity distribution. Using these optical elements, irradiation conditions of the pulsed light, including irradiation shape, light density and irradiation direction to the object, can be freely set.

<<Light Irradiation Unit 106 (Rotation Mechanism 108)>>

The light irradiation unit 106 emits measurement light onto an object. The light irradiation unit 106 may be integrated with the light source or may be connected to the light source via the optical member including the lens, the mirror, the diffusion plate and the optical fiber. In this embodiment, the light source 104, the light transmission unit 105 and the light irradiation unit 106 are interconnected.

The rotation mechanism 108 rotates the light irradiation unit 106. The center of the rotation is the center of the light emitting end of the light irradiation unit 106. The rotation mechanism 108 has a configuration such that the emission direction of the measurement light, which is emitted from the light irradiation unit 106, can be changed by rotating the rotation mechanism 108.

<<Scanning Mechanism 107>>

The scanning mechanism 107 moves the light irradiation unit 106 along the object 101. The scanning mechanism 107 can move the light irradiation unit 106 in the vertical direction in FIG. 1A, that is, the insertion direction of the object, and in the direction perpendicular to the paper surface. In other words, the light irradiation unit 106 can be moved by the scanning mechanism 107 in two-dimensional directions. The position of the light irradiation unit 106 on the scanning mechanism 107 is controlled by the control unit 109, which is described later. By combining the scanning mechanism 107 and the control unit 109, the photoacoustic measurement can be performed while moving the light irradiation unit two dimensionally for scanning. The vertical direction in FIG. 1A is the first axis according to the present invention, and a direction that is perpendicular to the paper surface, intersecting the first axis orthogonally, is the second axis according to the present invention.

<<Control Unit 109>>

The control unit 109 controls the position of the light irradiation unit 106 by driving the scanning mechanism 107. The control unit 109 also controls the direction of the measurement light emitted from the light irradiation unit 106 by driving the rotation mechanism 108. In order to increase intensity of the photoacoustic signal generated around the chest wall, the quantity of the measured light that is irradiated around the chest wall must be increased. Therefore the control unit 109 changes the emission direction of the measurement light, which is irradiated from the light irradiation unit 106, in accordance with the position of the light irradiation unit 106 (a position on the first axis according to the present invention). A concrete method to change the direction of the measurement light is described later.

<<Object 101 (Light Absorber 111)>>

The object 101 and the light absorbers 111a and 111b (collectively called “light absorber 111”) are not composing elements of the present invention, but will be described hereinbelow. The object 101 is a target of the photoacoustic measurement and typically is an organism. Here it is assumed that the object is a human breast.

In the photoacoustic measurement apparatus according to this embodiment, a light absorber 111 having a large light absorption coefficient existing inside the object 101 can be imaged. If the object is an organism, the light absorber 111 is, for example, water, lipids, melanin, collagen, protein, oxyhemoglobin or deoxyhemoglobin. The photoacoustic measurement apparatus according to this embodiment can perform angiography, diagnosis of malignant tumors and vascular diseases of humans and animals, and follow up after chemotherapy.

<<Holding Member 103>>

The holding member 103 holds the object 101 and is constituted by two holding members 103a and 103b (hereafter called “movable holding member 103a” and “fixed holding member 103b” respectively). Out of the two holding members, the fixed holding member 103b, where the probe is disposed, is secured to the breast, but the movable holding member 103a, where the light irradiation unit is disposed, can move independently from the light irradiation unit 106 so as to compress the breast.

To acoustically couple the probe and the object, it is preferable that the material of the holding member 103 has an acoustic impedance that is similar to that of the object. However if the object is held between two holding members and light is irradiated onto the surface of the object on the opposite side of the probe, as in the case of this embodiment, it is not necessary to consider the acoustic impedance for the movable holding member 103a on the side of the irradiating light, and any material of which transmittance is high with respect to the measurement light can be used. Typically a plastic plate (e.g. acrylic plate), a glass plate, polymethlpentene or the like can be used.

<<Probe 113>>

The probe 113 converts an acoustic wave (typically an ultrasound wave) generated inside the object 101 into an analog electric signal. The probe 113 may be a standalone acoustic detector or may be constituted by a plurality of acoustic detectors. The probe 113 may be a plurality of reception elements which are arrayed one dimensionally or two dimensionally. If multi-dimensional array elements are used, the measurement time can be decreased since the acoustic wave can be received at a plurality of locations simultaneously. If the probe is smaller than the object, the probe may scan the object so that the acoustic wave can be received at a plurality of locations. The light irradiation unit 106 and the probe 113 may be disposed so that the object is located therebetween, as in the case of this embodiment, or may be disposed on the same side of the object.

It is preferable that the probe 113 has high sensitivity and a wide frequency band. In concrete terms, piezoelectric ceramics (PZT), polyvinylidene fluoride (PVDF), capacitive micro-machine ultrasonic transducer (cMUT), a Fabry-Perot interferometer or the like can be used. The probe 113 is not limited to the examples mentioned here, but can be anything as long as the functions of a probe are satisfied.

The probe 113 must acoustically couple with the object 101 (and the fixed holding member 103b) in order to eliminate the influence of reflection and the attenuation of acoustic waves. For example, it is preferable to dispose an acoustic matching material, such an acoustic matching agent of water or oil between the probe 113 and the fixed holding member 103b. In this embodiment, the acoustic matching agent 114 is disposed between the probe 113 and the fixed holding member 103b.

<<Processing Unit 115>>

The processing unit 115 amplifies an electric signal acquired by the probe 113, converts the electric signal into a digital signal, and processes the digital signal to generate an image. The processing unit 115 generates an image to indicate an initial sound pressure distribution originated from the light absorber in the object, and an image to indicate an absorption coefficient distribution. The processing unit 115 may be a computer that includes a CPU, a main storage device and an auxilliary storage device, or may be specially designed dedicated hardware.

<Overview of Measurement Processing>

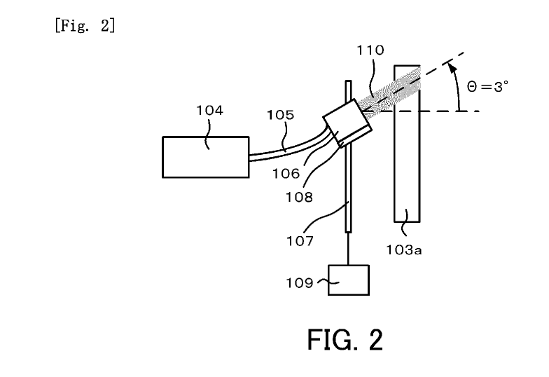

Overview of the measurement processing performed by the photoacoustic measurement apparatus according to Embodiment 1 will now be described with reference to FIG. 1A, FIG. 1B and FIG. 2. FIG. 1A shows a case when the light irradiation unit 106 is distant form the chest wall, and FIG. 1B shows a case when the light irradiation unit 106 is located close to the chest wall. FIG. 2 is an enlarged view of an area around the light irradiation unit 106.

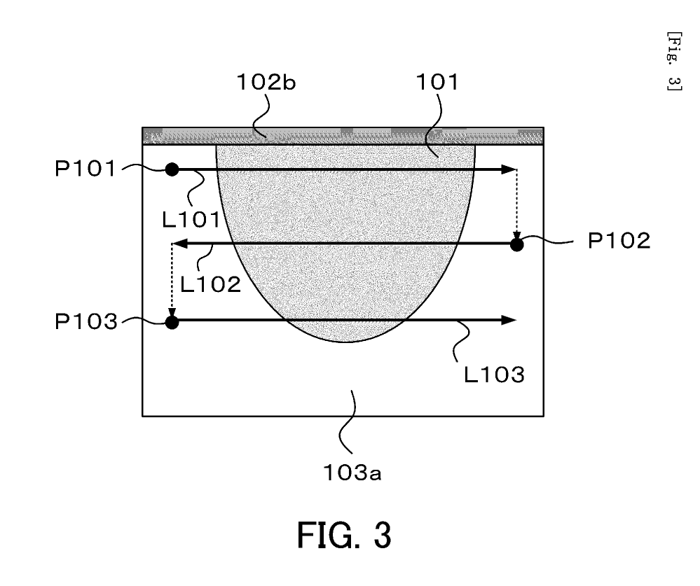

FIG. 3 shows a scanning route of the light irradiation unit 106 when the object 101 is viewed from the light irradiation unit 106 side. Here L101 to L103 are the scanning lines, and P101 to P103 are the start points of the scanning lines respectively. There are three scanning lines in this example, but the number of scanning lines is determined depending on the size of the object, the width of the probe or the like, and is not necessarily three.

The position of the light irradiation unit 106 on the scanning route is controlled by the control unit 109. In this embodiment, only when the light irradiation unit 106 is above L101, the control unit 109 drives the rotation mechanism 108 and inclines the light irradiation unit 106 toward the chest wall by 3 degrees. When the light irradiation unit 106 is above the other scanning lines (L102 and L103), on the other hand, the control unit 109 turns the light irradiation unit 106 to the front, that is, to the direction perpendicular to the movable holding member 103a.

The measurement light 110 irradiated onto the object 101 propagates while diffusing inside the object 101, and a part of the measurement light 110 is absorbed by a light absorber, such as blood vessels (light absorbers 111a and 111b in the case of FIG. 1A and FIG. 1B). The light absorber which absorbed the light generates an acoustic wave (acoustic wave 112a and acoustic wave 112b in the case of FIG. 1A and FIG. 1B) due to the photoacoustic effect. The generated acoustic wave propagates inside the object 101, and a part of the acoustic wave reaches the probe 113 via the fixed holding member 103b. The acoustic wave received by the probe 113 becomes an electric signal, is transferred to the processing unit 115 and processed to be a desired data, such as an image to represent the light absorption coefficient.

<Measurement Processing Flow Chart>

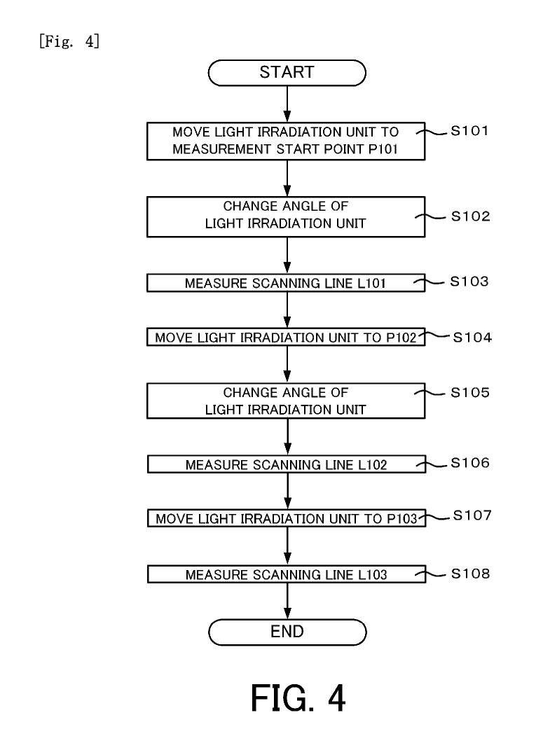

The flow to perform the above mentioned processing will now be described with reference to FIG. 4.

When the measurement starts, the control unit 109 drives the scanning mechanism 107 and moves the light irradiation unit 106 to a measurement start point P101 (S101). At this time, the light irradiation unit 106 is located above L101, hence the control unit 109 drives the rotation mechanism 108, and inclines the light irradiation unit 106 toward the chest wall by 3 degrees (S102).

When the processing in step S102 completes, the control unit 109 moves the light irradiation unit 106 along the scanning line L101, so as to perform measurement for the scanning line L101 (S103).

When the measurement for the scanning line L101 completes, the control unit 109 moves the light irradiation unit 106 to a start point P102 of the scanning line L102 (S104). At this time, the light irradiation unit 106 is located above L102, hence the control unit 109 drives the rotation mechanism 108 and returns the inclined light irradiation unit 106 to the original position (S105). Thereby the emission direction of the measurement light becomes perpendicular to the movable holding member 103a.

When the processing in step S105 completes, the control unit 109 moves the light irradiation unit 106 along the scanning line L102, and performs measurement for the scanning line L102 (S106).

When the measurement for the scanning line L102 completes, the control unit 109 moves the light irradiation unit 106 to a start point P103 of the scanning line L103 (S107). Then the control unit 109 moves the light irradiation unit 106 along the scanning line L103, and performs measurement for the scanning line L103 (S108).

According to Embodiment 1, the processing to incline the emission direction of the measurement light is performed only when the light irradiation unit 106 locates above the scanning line closest to the chest wall. Thereby a sufficient quantity of light can be irradiated onto an area near the chest wall, while minimizing the influence on the measurement result.

In Embodiment 1, the light irradiation unit 106 is inclined only when the light irradiation unit 106 locates above the scanning line L101, but another method may be used. For example, the inclination angle may be determined for each scanning line in accordance with the distance between the light irradiation unit 106 and the chest wall. The light irradiation unit 106 may be inclined only in a specific area while scanning a certain scanning line. Or the angle of the light irradiation unit 106 may be continuously changed during scanning. In any case, the present invention can solve the problem if only the direction of the measurement light emitted from the light irradiation unit 106 can be determined based on the distance between the chest wall support unit 102 and the light irradiation unit 106. It is preferable that the emission direction of the measurement light is positioned closer to the chest wall side (that is, the test subject support member side), and the incident angle to the object is decreased as the light irradiation unit 106 becomes closer to the upper end of the movable range thereof.

### Embodiment 2

In Embodiment 1, the emission direction of the measurement light is changed by rotating the light irradiation unit 106 itself using the rotation mechanism 108. In Embodiment 2, however, a mechanism to change the direction of the optical path is installed inside the light irradiation unit 106, and the emission direction of the measurement light is changed by driving this mechanism.

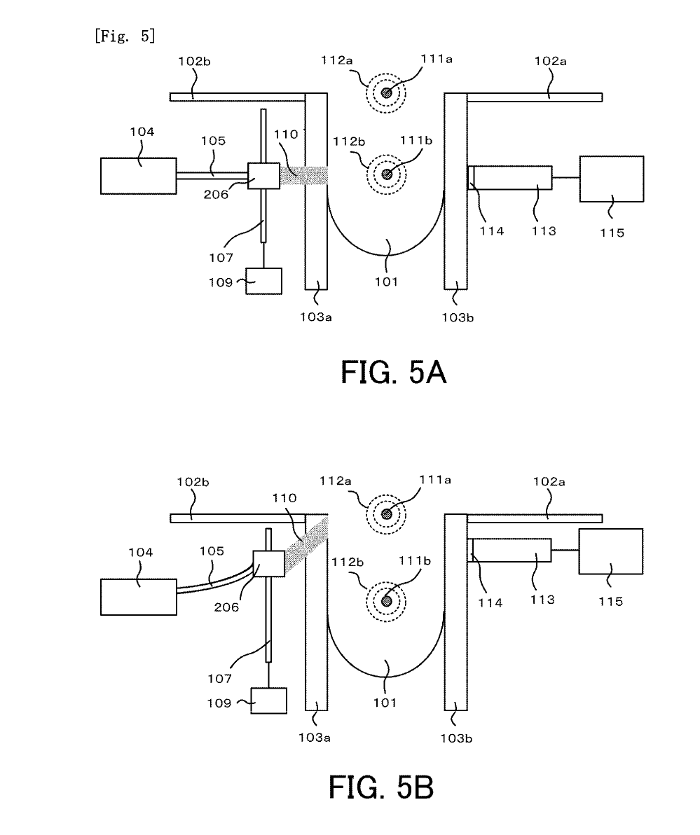

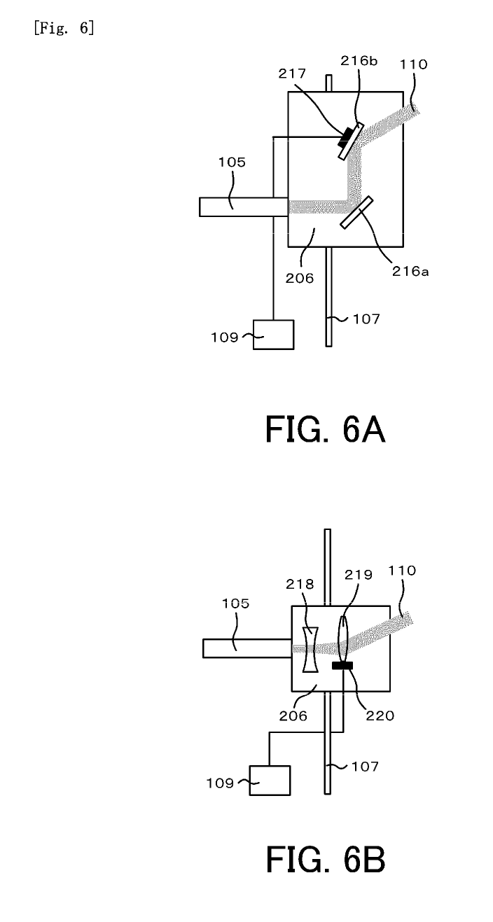

FIG. 5A, FIG. 5B, FIG. 6A and FIG. 6B are diagrams describing a photoacoustic measurement apparatus according to Embodiment 2. FIG. 5A shows a case when the light irradiation unit 106 is distant from the chest wall, and FIG. 5B shows a case when the light irradiation unit 106 is close to the chest wall. As a rule, a composing element the same as Embodiment 1 is denoted with a same reference number, and description thereof is omitted.

A difference of the light irradiation unit 206 according to Embodiment 2 from Embodiment 1 (light irradiation unit 106) is that an optical path switching mechanism is enclosed. In concrete terms, the emission direction of the measurement light is changed by rotating a light reflection member installed inside. FIG. 6A shows the internal structure of the light irradiation unit 206.

In FIG. 6A, the reference sign 216a is a first light reflection member, the reference sign 216b is a second light reflection member, and the reference sign 217 is a rotation mechanism. Typically the light reflection member is a mirror. In the second embodiment, the control unit 109 changes the emission direction of the measurement light by rotating the rotation mechanism 217.

The scanning route of the probe 113 and the processing flow according to Embodiment 2 are the same as Embodiment 1. In other words, if the light irradiation unit 206 is near the chest wall (above the scanning line L101), the emission direction of the measurement light is inclined toward the chest wall. If the light irradiation unit 206 is distant from the chest wall (above the scanning lines L102 and L103), the emission direction of the measurement light is set to be perpendicular to the movable holding member 103a. The only difference is that the rotation mechanism 217, not the rotation mechanism 108, is driven in step S102 and step S105.

Therefore in Embodiment 2, only the enclosed light reflection member rotates, not the entire light irradiation unit 206, hence this minimizes the physical restrictions caused by the peripheral components of the light irradiation unit 206.

In Embodiment 2, the emission direction of the measurement light is changed by rotating the rotation mechanism linked to the light reflection member 216b, but other methods may be used. For example, as shown in FIG. 6B, one concave lens 218 and one convex lens 219 may be disposed instead of the light reflection member, so that the optical axis is shifted from the center of the convex lens 219 by moving the moving mechanism 220 linked with the convex lens 219 in parallel. Thereby the direction of the measurement light emitted from the light irradiation unit 206 can be changed. Any mechanism can be used for the internal mechanism to change the emission direction of the measurement light.

### Embodiment 3

In Embodiment 1 and Embodiment 2, the emission direction of the measurement light that is emitted from the light irradiation unit 106 is determined for each scanning line. If this method is used, however, the quantity of light irradiated onto an area outside the chest wall drops on the scanning line closest to the chest wall. To solve this problem, in Embodiment 3, scanning is performed on a same scanning line for a plurality of times, while changing the emission direction of the measurement light.

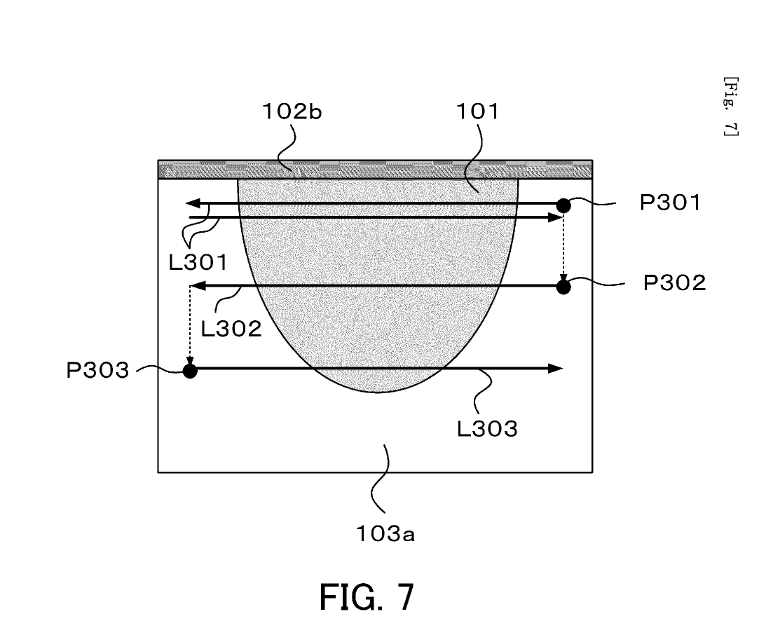

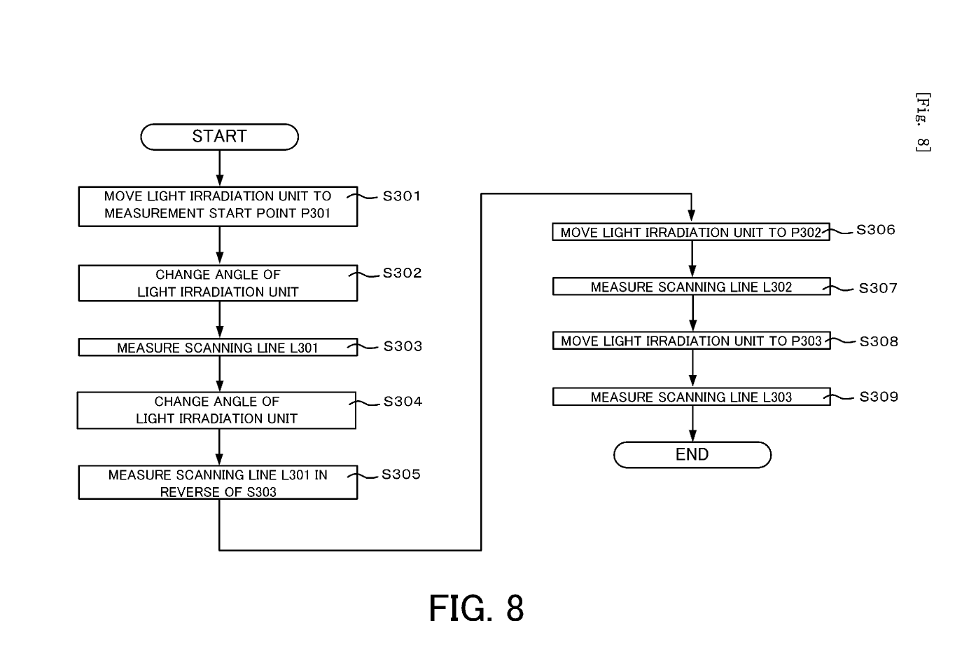

FIG. 7 is a diagram describing a scanning route according to Embodiment 3, and FIG. 8 is a flow chart depicting processing of a photoacoustic measurement apparatus according to Embodiment 3. The configuration of the photoacoustic measurement apparatus according to Embodiment 3 is the same as Embodiment 1.

The measurement flow according to Embodiment 3 will be described with reference to FIG. 8.

When the measurement starts, the control unit 109 moves the light irradiation unit 106 to a measurement start point P301 (S301). Then the control unit 109 drives the rotation mechanism 108, and inclines the light irradiation unit 106 toward the chest wall by 3 degrees (S302).

When the processing in step S302 completes, the control unit 109 moves the light irradiation unit 106 along the scanning line L301, so as to perform measurement for the scanning line L301 (S303).

When the first measurement for the scanning line L301 completes, the control unit 109 drives the rotation mechanism 108 to return the inclined light irradiation unit 106 to the original position (S304). Then the second measurement is performed for the scanning line L301 by scanning the scanning line L301 again (S305). In FIG. 7, two scanning lines L301 are illustrated for the purpose of description, but the scan areas are exactly the same.

When the second measurement for the scanning line L301 completes, the control unit 109 moves the light irradiation unit 106 to a start point P302 of the scanning line L302 (S306). Then the control unit 109 moves the light irradiation unit 106 along the scanning lien L302, and performs measurement for the scanning line L302 (S307).

When the measurement for the scanning line L302 completes, the control unit 109 moves the light irradiation unit 106 to a start point P303 of the scanning line L303 (S308). Then the control unit 109 moves the light irradiation unit 106 along the scanning line L303, and performs measurement for the scanning line L303 (S309).

Therefore according to Embodiment 3, for the same scanning line L301, scanning with changing the emission direction of the measurement light and scanning without changing the emission direction of the measurement light are executed in combination. Then information around the chest wall can be accurately acquired as in the other embodiments, and a sufficient quantity of measurement light can be irradiated, even on an area outside the area near the chest wall.

In Embodiment 3, scanning is performed for the scanning line L301 in a state where the light irradiation unit 106 is inclined, and then the light irradiation unit 106 is rotated to face the front, and scanning is performed again in reverse, but the sequence of scanning is arbitrary. For example, L301 to L303 may be scanned first without inclining the emission direction of the measurement light, and then L301 may be scanned again with inclining the emission direction of the measurement light. All that is required is to scan a same scanning line a plurality of times, and change the emission direction of the measurement light at least one of those times.

### EXAMPLE

An example corresponding to Embodiment 1 will now be described.

In this example, 3-mm thick tungsten carbide is used for the first chest wall support unit 102a and the second chest wall support unit 102b respectively. The breast, which is the object, is held between the movable holding plate 103a and the fixed holding plate 103b. For the light source 104, a wavelength-variable titanium-sapphire laser is used. The pulse width of the laser used here is 10 nanoseconds, the frequency is 10 Hz, and the wavelength is 797 nm.

The distance between the center of the light emission portion of the light irradiation unit 106 and the upper surface of the chest support unit, when the light irradiation unit 106 is above the scanning line L101, is 33 mm. The distance between the surface where the object 101 and the movable holding member 103a contact, and the light emitting end of the light irradiation unit 106, is 165 mm. In order to efficiently receive the acoustic wave from the object, a 20-mm thick movable holding member made of acrylic is used for the movable holding member 103a.

A 7-mm thick polymethlpentene member is used for the fixed holding member 103b. A piezoelectric probe made of lead zirconate titanate (PZT) is used for the probe 113. For acoustic matching between the fixed holding member 103b and the probe 113, an acoustic matching agent 114 (caster oil) is disposed between the fixed holding member 103b and the probe 113.

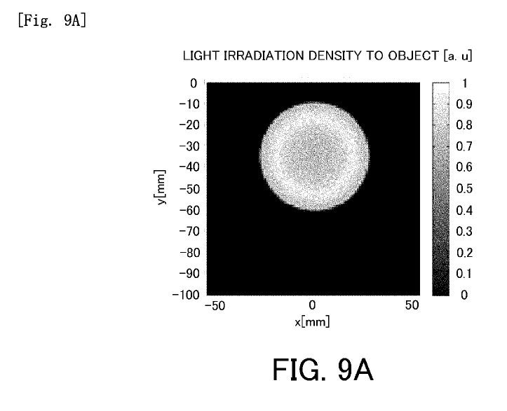

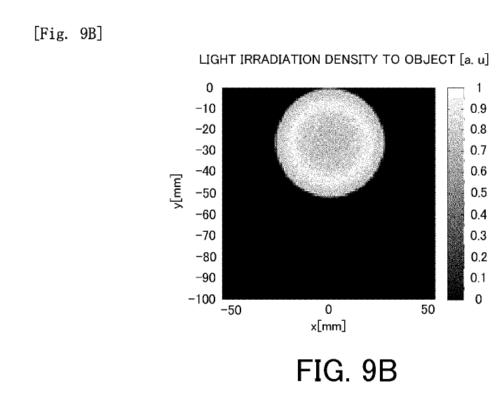

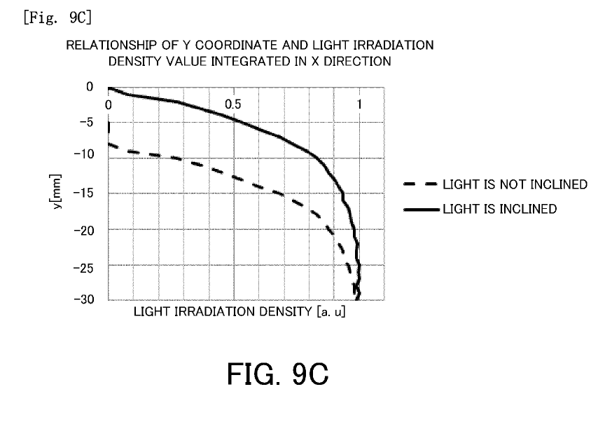

FIG. 9A shows a distribution of the light irradiation density of light irradiated onto the object when the light irradiation unit 106 is above the scanning line L101 and the measurement light is in a direction perpendicular to the movable holding member 103a. The X axis in FIG. 9A is a direction perpendicular to the paper surface in FIG. 1A, and the origin is the center of the breast. The Y axis in FIG. 9A is a direction perpendicular to the second chest wall support unit 102b, and the origin is the support surface of the second chest wall support unit. FIG. 9B shows a distribution of the light irradiation density of light irradiated onto the object when the light irradiation unit 106 is above the scanning line L101, and the measurement light is inclined toward the chest wall by 3 degrees.

FIG. 9C shows values when the light irradiation density on the Y coordinate is integrated in the X direction for FIG. 9A and FIG. 9B respectively, where the ordinate is the Y coordinate and the abscissa is a value when the light irradiation density is integrated in the X direction. As FIG. 9C shows, the quantity of measurement light that is irradiated onto an area around the chest wall can be secured by inclining the light irradiation unit 106 toward the chest wall.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2013-043337, filed on Mar. 5, 2013, which is hereby incorporated by reference herein in its entirety.

### REFERENCE SIGNS LIST

104: Light source, 105: Light transmission unit, 106: Light irradiation unit, 107: Scanning mechanism, 109: Control unit, 113: Probe, 115: Processing unit

1. An object information acquiring apparatus, comprising:
a light irradiation unit that irradiates light onto an object and can change an emission direction of the light;
a scanning mechanism that moves said light irradiation unit along a first axis;
a probe that receives an acoustic wave generated by light irradiated onto the object;
a processing unit that generates characteristic information about a region inside the object based on the acoustic wave received by said probe; and
a control unit that controls a position of said light irradiation unit on said first axis, and the emission direction of the light emitted from said light irradiation unit,
wherein said control unit determines the emission direction of the light emitted from said light irradiation unit based on the position of said light irradiation unit on said first axis.

2. The object information acquiring apparatus according to claim 1, wherein said control unit controls the emission direction of the light so that an incident angle of the light incident on the object decreases as the position of said light irradiation unit on said first axis becomes closer to an edge of a movable range of said light irradiation unit.

3. The object information acquiring apparatus according to claim 1, further comprising a test subject support member that has an opening in which to insert the object,
wherein said first axis is parallel to an insertion direction along which the object is inserted into said opening, and
wherein said control unit performs control so that the emission direction of the light turns more toward said test subject support member as the position of said light irradiation unit on said first axis becomes closer to said test subject support member.

4. The object information acquiring apparatus according to claim 1,
further comprising a rotation mechanism configured to rotate said light irradiation unit, and
wherein said control unit changes the emission direction of the light by rotating said rotation mechanism.

5. The object information acquiring apparatus according to claim 1,
wherein said light irradiation unit includes an optical path switching mechanism, and
wherein said control unit changes the emission direction of the light by means of said optical path switching mechanism.

6. The object information acquiring apparatus according to claim 5, wherein said optical path switching mechanism is a mechanism that includes a rotatable light reflection member, and said control unit changes the emission direction of the light by rotating said light reflection member.

7. The object information acquiring apparatus according to claim 5, wherein said optical path switching mechanism is a moving mechanism to move at least one lens, and said control unit changes the emission direction of the light by moving said lens.

8. The object information acquiring apparatus according to claim 1, wherein said scanning mechanism has a configuration that allows movement of said light irradiation unit along a second axis which is perpendicular to said first axis, and
wherein said control unit executes the scanning at least partly by moving said light irradiation unit along said second axis a plurality of times, and determines the emission direction of the light emitted from said light irradiation unit based on the position of said light irradiation unit on said first axis at least once out of the plurality of times of scanning.

9. A control method of an object information acquiring apparatus including a light irradiation unit that irradiates light onto an object and can change an emission direction of the light, and a probe that receives an acoustic wave generated by light irradiated onto the object, the control method comprising:
a moving step of moving the light irradiation unit along a first axis;
a control step of determining the emission direction of the light based on a position of the light irradiation unit on the first axis;
a receiving step of receiving an acoustic wave by the probe; and
a processing step of generating characteristic information about inside the object based on the received acoustic wave.

10. The control method of an object information acquiring apparatus according to claim 9, wherein, in said control step, the emission direction of the light is determined so that an incident angle of the light to the object decreases as the position of the light irradiation unit on the first axis becomes closer to an edge of a movable range of the light irradiation unit.

11. The control method of an object information acquiring apparatus according to claim 9, wherein the object information acquiring apparatus further includes a test subject support member that has an opening into which to insert the object, and the first axis is parallel to an insertion direction in which the object is moved for insertion into the opening, and
wherein, in said control step, control is performed so that the emission direction of the light turns more toward the test subject support member as the position of the light irradiation unit on the first axis becomes closer to the test subject support member.

12. The control method of an object information acquiring apparatus according to claim 9, wherein the light irradiation unit can be rotated by a rotation mechanism, and
wherein, in said control step, the emission direction of the light is changed by rotating the rotation mechanism.

13. The control method of an object information acquiring apparatus according to claim 9, wherein the light irradiation unit includes an optical path switching mechanism, and
wherein, in said control step, the emission direction of the light is changed by the optical path switching mechanism.

14. The control method of an object information acquiring apparatus according to claim 13, wherein the optical path switching mechanism is a mechanism that includes a rotatable light reflection member, and
wherein, in said control step, the emission direction of the light is changed by rotating the light reflection member.

15. The control method of an object information acquiring apparatus according to claim 13, wherein the optical path switching mechanism is a moving mechanism to move at least one lens, and
wherein, in said control step, the emission direction of the light is changed by moving the lens.

16. The control method of an object information acquiring apparatus according to claim 9, wherein, in said moving step, the light irradiation unit is further moved along a second axis which is perpendicular to the first axis, and
in said control step, the scanning with moving the light irradiation unit along the second axis is executed a plurality of times, and the emission direction of the light emitted from the light irradiation unit is determined based on the position of the light irradiation unit on the first axis at least once out of the plurality of times of scanning.
